(19) United States
(12) Patent Application Publication
(10) Pub. No.: US 2008/0220050 A1
(43) Pub. Date: Sep. 11, 2008

(54) Compositions For Gingival Retraction And Other Methods

(75) Inventors: Xiangxu Chen, Diamond Bar, CA (US); Xuejun Qian, Foothill Ranch, CA (US)

Correspondence Address: WOOD, HERRON & EVANS, LLP, 2700 CAREW TOWER, 441 VINE STREET, CINCINNATI, OH, 45202, US

(73) Assignee: KERR CORPORATION, Orange, CA (US)

(21) Appl. No.: 11/683,644

(22) Filed: Mar. 8, 2007

(51) Int. Cl. A61K 33/06

(52) U.S. Cl. 424/434; 514/772

(57) ABSTRACT

At least one clay, at least one glass filler, and at least one astringent agent are formulated with water into a paste for injection in dental and medical applications. In one embodiment, the paste is used to treat and/or widen a gingival sulcus. The paste is readily rinsed off the surface to which it is applied using water.

### FIELD OF THE INVENTION

Compositions for gingival retraction in dental and/or medical treatments.

### BACKGROUND

The gingiva, composed of mucosa, is the soft tissue that connects teeth and bone. It is a common practice for dental practitioners to temporarily widen a gingival sulcus for further dental treatments, such as impressions.

Several methods to temporarily widen a gingival sulcus have been developed. These methods may be classified as mechanical methods that involve placement of a string into the gingival sulcus to physically displace the tissue; chemo-mechanical methods that involve treatment with one or more chemicals that may shrink the tissues temporarily and may also control hemorrhage; rotary curettage methods; and electro-surgery methods.

An astringent agent is a chemical that tends to shrink or constrict body tissues. This effect is usually local after topical application. Astringents have been widely used in gingival retraction procedures to stop bleeding or oozing. Astringent chemicals that are commonly used in the chemo-mechanical method may be alums, aluminum chloride, aluminum sulfate, ferric chloride, ferric sulfate, zinc chloride, zinc sulfate, and epinephrine, among which aluminum chloride, ferric sulfate and epinephrine are the most widely applied.

Gingival retraction cords, such as Ultrapak (Ultradent, South Jordan Utah) have been commercialized. During gingival retraction procedures, gingival reaction cords are packed and maintained between the gingiva and tooth, then are removed before further dental treatments. Dental practioners generally find retention cord packing a time-consuming and frustrating procedure. Bleeding and oozing may also result from pressure applied during the packing procedure.

Dental tools have been developed to facilitate gingival retraction. Laser may be used to treat gingival tissues together with other treatments. Lasers can promote healing of gums, reattach gum tissues to root surfaces, and destroy bacteria involved in gum diseases.

Such procedures are time-consuming and require skills. The above factors are exacerbated when gingival retractions are applied on several teeth at the same time.

Cordless mechanochemical gingival retraction materials have been developed, and astringent chemicals may be included to effectively cause tissue or blood vessel to contract to further control oozing of gingival tissue.

Other compositions and methods are desirable.

### SUMMARY

One embodiment is a gingival retraction composition comprising (a) a clay, (b) a micronized glass filler, (c) an astringent agent, and (d) water, the composition forming a viscous paste.

Another embodiment is a method that controls bleeding while temporarily widening a gingival sulcus. In the method, a paste comprising a clay, a micronized glass filler, an astringent agent, and water is inserted within a gingival sulcus. The paste is allowed to remain in the sulcus from about one second to about 15 minutes, and is then rinsed off with water.

These and other embodiments will be appreciated with reference to the following description and examples.

### DETAILED DESCRIPTION

Compositions for gingival retraction in dental and/or medical applications. In one embodiment, the compositions are injectable and are used to treat and/or widen a gingival sulcus by injection. The composition may be rinsed off by water after application. The compositions are biocompatible, hydrophilic, and hemostatic. The composition contains at least one clay. The composition also contains at least one micronized glass filler, at least one astringent agent, and water. In one embodiment, at least one clay, at least one micronized glass filler, and at least one astringent agent are the only components that materially affect the composition. In one embodiment, at least one clay, at least one micronized glass filler, at least one astringent agent, and water are the only components that materially affect the composition. In any embodiment, the addition of water renders the composition into a paste.

Clays are naturally occurring fine-grain particles in sediment, soil, or rock, consisting of a variety of phyllosilicate minerals rich in silicon, aluminum oxides, hydroxides, and a variety of structural water. Clays are distinguished from other small particles present in sediment/soil/rock, such as silt and sand, by their small size, flake or layered shape, affinity for water, and high plasticity. Clays may have high plasticity when mixed with certain amounts of water.

Clay minerals are known to include the following groups: kaolinite, smectite, illite, and chlorite. Kaolinites include the minerals kaolinite, dickite, halloysite, and ancrite. Smectites include pyrophyllite, talc, vermiculite, sauconite, saponite, nontronite, and montmorillonite. Illites include micas. Chlorites include a variety of similar minerals with considerable chemical variation. Clays of kaolinite and smectite groups are used for skin care applications.

Montmorillonite is a very soft mineral of the smectite group. It has two tetrahedral sheets sandwiching a central octahedral sheet, which is also known as a 2:1 clay. Kaolinite has one tetrahedral sheet linked through oxygen atoms to one octahedral sheet of alumina octahedral, also known as a 1:1 clay. Bentonite is a clay consisting mostly of montmorillonite. Bentonite and montmorillonite are sometimes used interchangably to refer to the same mineral. Two types of bentonites exist: sodium bentonite (swelling bentonite) and calcium bentonite (non-swelling bentonite). Bentonites are formed from hydrothermal weathering of volcanic ash.

The clay can be a sheet clay, which includes kaolinite, montmorillonite (bentonite), talc, mica (illite), serpentine, chlorite, mullite, kyanite, pumice, goethite, and/or pyrophyllite. In one embodiment, the clay is kaolinite and/or bentonite. In one embodiment, the clay is micronized kaolinite and bentonite. The clay concentration in the composition may be between 1 wt % and 80 wt % inclusive. In one embodiment, the clay concentration in the composition may be between 10 wt % and 50 wt % inclusive.

The clay has plasticity when it is mixed with a volume of water to form a paste. The concentration of water in the composition may be between about 1 wt % and 50 wt %. In one embodiment, the concentration of water in the composition may be between about 5 wt % and 30 wt %.

Glass is a type of uniform amorphous solid inorganic substances, formed by heating a mixture of minerals, sands, and other inorganic materials. Common glass contains a significant amount of silicon dioxide. Glass surfaces are generally hydrophilic and may be wetted by water. Glass is generally considered as a biologically inactive material. With special treatments, such as the addition of other compounds or heat treatment, glass will not break into sharp shards. The mixture of micronized glass particles and water do not form pastes with good plasticity and may be dispersed into large amount of water.

Silicate is the largest group of minerals. Chemically, silicate is a compound that contains an anion in which one or more central silicon atoms are surrounded by electronegative ligands. Silicon dioxide, also named as silica and including quartz, may also be considered as a silicate, although there is no negative charge and no need for counter-ions. Borosilicate glass is a particular type of glass that contains a significant amount of boric oxide. Compared with many other glasses, borosilicate glass has superior durability, and chemical and heat resistance.

Water soluble organic agents may be mixed into the composition to adjust its viscosity. Water soluble organic agents include, but are not limited to, ethanol, isopropylene alcohol, acetone, citric acid, sodium citrate, pentaerythritol, ethoxylated pentaerythritol, glycerin, ethoxylated glycerin, ethylene glycol, poly(ethylene glycol), propylene glycol, and/or poly(propylene glycol).

The presence of a micronized glass filler renders the final paste mixture more easily rinsed off by water generated from a dental apparatus. In one embodiment, the average particle size of the micronized glass filler ranges from about 0.05 μm to about 100 μm. In one embodiment, the average particle size of the micronized glass filler ranges from about 0.1 μm to about 50 μm. In one embodiment, the average size of the micronized glass filler ranges from about 0.5 μm to about 20 μm. In one embodiment the glass filler is a combination of amorphous inorganic substances based on silicates that include, but are not limited to, silicon dioxide, nesosilicates, sorosilicates, cyclosilicates, and phyllosilicates. In one embodiment, a micronized glass filler is an inorganic substance that contains boron. In one embodiment, the micronized glass filler is a borosilicate glass. In one embodiment, the micronized glass filler is a PYREX® glass. The micronized glass filler may be mixed with a certain volume of water to provide some plasticity to the resulting paste. In one embodiment, the plasticity of the glass filler and water mixture is equal to or less than the mixture of the clay and water mixture. In one embodiment, the concentration of the glass filler is between about 1 wt % and about 80 wt %, inclusive. In one embodiment, the concentration of the glass filler is between about 20 wt % and about 70 wt %, inclusive.

One or more astringent agent, also referred to as astringent, may also be incorporated in the composition. Astringents include, but are not limited to, alums, aluminum chloride, aluminum sulfate, ferric chloride, ferric sulfate, zinc chloride, zinc sulfate and epinephrine. In one embodiment, astringents incorporated in the composition include aluminum chloride, ferric sulfate, and/or epinephrine. In one embodiment, the concentration of astringent is between about 0.1 wt % and about 30 wt %, inclusive. In one embodiment, the concentration of astringent is between about 10 wt % and about 20 wt %, inclusive.

In one embodiment, the viscosity of the paste is measured by a dynamic stress rheometer and is higher than about 13000 Pascals/second. The viscosity of the paste in the present invention can be determined using a penetrometer, or other methods known to one skilled in the art.

In one embodiment, a universal penetrometer is used to measure viscosities of a wide variety of materials using penetration of weighted needles. A plunger is released to penetrate into viscous pastes and depth of penetrations are used to compare viscosities. A Precision 73515 (Houston Tex.) universal penetrometer is employed to evaluate paste viscosities using American Society for Testing and Materials (ASTM) D-5. The total weight of the plunger rod and the penetrating needle is 50 grams and extra weight may be added to bring the total weight of penetration to 100 grams and 150 grams. The diameter of the penetrating needle is 1 mm. The duration of penetrations is set to be 10 seconds. The sample container has a diameter of 10 mm and a depth of 8 mm. Three penetrations may be applied on each freshly prepared sample at 24° C.±1° C. Using the penetrometer, the probe without additional weight penetrates about 1.1 mm on EXPASYL® (Kerr, Orange Calif.). In one embodiment, the penetration depth without additional load on the disclosed composition is between about 0.01 mm to about 7.5 mm. In one embodiment, the penetration depth is between about 0.05 mm to about 3 mm. In one embodiment, the penetration depth is between about 0.1 mm to about 2 mm.

A pH buffering agent may be included in the gingival retraction material to make the composition less acidic and hence more biocompatible. Buffering agents include, but are not limited to, sodium bicarbonate, sodium carbonate, potassium bicarbonate, and/or potassium carbonate. In one embodiment, the concentration of pH buffering agent is between about 0.01 wt % to about 10 wt %. In one embodiment, the concentration of astringent agent is between about 0.1 wt % and about 5 wt %.

A flavorant may be included to make the material have a more desirable taste and smell. Flavorants include, but are not limited to, citrus (e.g., orange, lime), mints (e.g., peppermint), isoamyl acetate, ethyl propionate, and/or ethyl maltol. In one embodiment where a flavorant is included, the concentration of flavorant is between about 0.0001 wt % and about 5 wt % inclusive. In one embodiment where a flavorant is included, the concentration of flavorant is between about 0.001 wt % and about 2 wt % inclusive.

A colorant may be included to introduce a distinctive color to the composition. Colorants include, but are not limited to, dyes, pigments, and inks. In one embodiment, food dyes are used which include, but are not limited to, Brilliant Blue FCF, indigotine, Fast Green FCF, Allura Red AC, tartrazine, and/or Orange Yellow S. In one embodiment where a colorant is used the concentration of colorant is between about 0.0001 wt % and about 3 wt % inclusive. In one embodiment where a colorant is used, the concentration of colorant is between about 0.001 wt % and about 1 wt % inclusive.

The composition may be inserted into the gingival sulcus by various methods that include, but are not limited to, an injection device. In one embodiment, the composition is injected into the gingival sulcus using a device having a needle with a diameter between about 0.2 mm and 2 mm that contacts gingival tissue. In one embodiment, the composition is injected into the gingival sulcus using a device having a needle with a diameter between about 0.7 mm and 1.6 mm that contacts gingival tissue. Other diameter needles may be determined by actual applications. In one embodiment, the gingival retraction composition remains in the sulcus for about one second to about 15 minutes. In one embodiment, the gingival retraction composition remains in the sulcus for about ten seconds to about five minutes. Due to the high viscosity of the paste, in one embodiment the gingival sulcus is widened to obtain a retraction effect. Multiple injections may be needed to achieve desired retraction. In one embodiment, gingival tissue bleeding is controlled by the astringent agent in the composition.

After widening the gingival sulcus, the gingival retraction composition is then rinsed off by water generated from a dental apparatus.

The composition may also be used after widening the gingival sulcus using other methods. Such methods include, but are not limited to, rotary curettage and electro-surgery methods to reduce bleeding or oozing and maintain the widened gingival sulcus. The composition may also be used to control bleeding after cavity preparation prior to further dental restorative procedures.

The following examples illustrate embodiments and uses of the composition, and do not limit the scope of the disclosure.

### EXAMPLE 1

| | |
|---|---|
| Bentonite | 47.1% |
| Aluminum chloride | 23.5% |
| Water | 29.4% |

Using a penetrometer, the probe without additional weight penetrated about 0.2 mm. About 0.1 g of the above composition was prepared in a ball shape. Ten g of water was added, followed by vigorous shaking. The total time for the paste to fully disperse into the water was about two and one-half minutes.

### EXAMPLE 2

| | |
|---|---|
| Bentonite | 19.6% |
| Aluminum chloride | 17.4% |
| Water | 19.6% |
| Micronized borosilicate | 44.4% |
| glass | |

Using a penetrometer, the probe without additional weight penetrated about 0.6 mm. About 0.1 g of the above composition was prepared in a ball shape. Ten g of water was added, followed by vigorous shaking. The total time for the paste to fully disperse into the water was 1 about minute and 40 seconds.

### EXAMPLE 3

| | |
|---|---|
| Bentonite | 43.8% |
| Aluminum chloride | 25.0% |
| Water | 31.3% |

Using a penetrometer, the probe without additional weight penetrated about 1.2 mm. About 0.1 g of the above composition was prepared in a ball shape. Ten g of water was added, followed by vigorous shaking. The total time for the paste to fully disperse into water was about two and one-half minutes.

### EXAMPLE 4

| | |
|---|---|
| Bentonite | 13.5% |
| Aluminum chloride | 15.4% |
| Water | 17.3% |
| Micronized borosilicate | 53.9% |
| glass | |

Using a penetrometer, the probe without additional weight penetrated about 1.6 mm. About 0.1 g of the above composition was prepared in a ball shape. Ten g of water was added, followed by vigorous shaking. The total time for the paste to fully disperse into water was about 1 minute and 35 seconds.

### EXAMPLE 5

| | |
|---|---|
| Bentonite | 52.4% |
| Aluminum chloride | 19.1% |
| Water | 28.6% |

Using a penetrometer, the probe without additional weight penetrated about 0.5 mm. About 0.1 g of the above composition was prepared in a ball shape. Ten g of water was added, followed by vigorous shaking. The total time for the paste to fully disperse into water was about 3 minutes and 40 seconds.

### EXAMPLE 6

| | |
|---|---|
| Bentonite | 15.8% |
| Aluminum chloride | 15.8% |
| Water | 15.8% |
| Micronized borosilicate | 52.6% |
| glass | |

Using a penetrometer, the probe without additional weight penetrated about 0.8 mm. About 0.1 g of the above composition was prepared in a ball shape. Ten g of water was added, followed by vigorous shaking. The total time for the paste to fully disperse into water was about 1 minute and 35 seconds.

Other variations or embodiments will also be apparent to one of ordinary skill in the art from the above description and examples. As one example, the clay, glass filler, and astringent agent components of the composition may be provided in a kit, with instructions to add water to form a paste, for use of the composition, etc. As another example, a device for insertion of the composition into a gingival sulcus may be included with a kit. Thus, the forgoing embodiments are not to be construed as limiting the scope of the following claims.

What is claimed is:

1. A gingival retraction comprising (a) a clay, (b) a micronized glass filler, (c) an astringent agent, and (d) water forming a viscous paste gingival retraction composition.

2. The composition of claim 1 wherein the clay is selected from at least one of kaolin, bentonite, illite, or chlorite.

3. The composition of claim 1 wherein the clay is present in a concentration ranging from about 1 wt % to about 80 wt % inclusive.

4. The composition of claim 1 wherein the glass filler is a borosilicate glass filler.

5. The composition of claim 1 wherein the glass filler is present in a concentration ranging from about 1 wt % to about 80 wt % inclusive.

6. The composition of claim 1 wherein the average particle size of the micronized glass filler ranges from about 0.05 μm to about 100 μm.

7. The composition of claim 1 wherein the concentration of the astringent agent ranges from about 0.1 wt % to about 30 wt %.

8. The composition of claim 1 wherein the astringent agent is selected from the group consisting of alums, aluminum chloride, aluminum sulfate, ferric chloride, ferric sulfate, zinc chloride, zinc sulfate, epinephrine, and combinations thereof.

9. The composition of claim 1 further comprising a buffering agent.

10. The composition of claim 9 wherein the buffering agent is present in a concentration ranging from about 0.01 wt % to about 10 wt %.

11. The composition of claim 9 wherein the buffering agent is selected from the group consisting of sodium bicarbonate, sodium carbonate, potassium bicarbonate, potassium carbonate, and combinations thereof.

12. The composition of claim 1 further comprising a flavorant.

13. The composition of claim 12 wherein the flavorant is present in a concentration ranging from about 0.0001 wt % to about 5 wt %.

14. The composition of claim 12 wherein the flavorant is selected from the group consisting of citrus, mint, and combinations thereof.

15. The composition of claim 1 further comprising a colorant.

16. The composition of claim 15 wherein the colorant is present at a concentration ranging from about 0.0001 wt % to about 3 wt %.

17. The composition of claim 1 wherein penetration into the paste is between about 0.05 and about 3 mm, using ASTM D-5 with total weight of the plunger and needle of 50 grams, test duration of 10 seconds, and samples size of 10 mm in diameter and 8 mm in depth.

18. A method for temporarily widening a gingival sulcus with controlled bleeding, the method comprising inserting a paste within a gingival sulcus, allowing the paste to remain in the sulcus from about one second to about 15 minutes, and rinsing off the paste with water, the paste comprising a clay, a micronized glass filler, an astringent, and water, to result in a temporarily widened gingival sulcus with controlled bleeding.

19. The method of claim 18 wherein the paste is inserted into the gingival sulcus through a device with a needle.

20. The method of claim 18 wherein the paste further comprises at least one component selected from the group consisting of a buffering agent, a flavorant, and a colorant.

21. A gingival retraction composition consisting essentially of (a) a clay, (b) a micronized glass filler, and (c) an astringent agent, where water is added to form a viscous paste gingival retraction composition.
